(19) United States
(12) Patent Application Publication
(10) Pub. No.: US 2026/0191681 A1
(43) Pub. Date: Jul. 9, 2026

(54) INJECTOR FOR AN IMPLANT

(71) Applicant: IMSTec GmbH, Klein-Winternheim (DE)

(72) Inventor: Edgar Mähringer-Kunz, Münster-Sarmsheim (DE)

(21) Appl. No.: 19/442,170

(22) Filed: Jan. 7, 2026

(30) Foreign Application Priority Data
Jan. 7, 2025 (DE) 10 2025 000 049.9

(51) Int. Cl. A61F 9/00

(52) U.S. Cl. CPC A61F 9/0017; A61F 2240/001; A61F 2250/0067

(57) ABSTRACT

An injector for an implant, wherein the implant is stored in the injector, wherein the injector has an injection opening for delivering the implant into an organ, in particular an eye, a storage portion in which the implant is stored, and a feed mechanism for ejecting the implant from the storage portion through the injection opening. It is proposed that the implant is retained in the storage portion by means of a holding element which makes contact with the implant.

### CROSS-REFERENCE TO RELATED APPLICATIONS

This application claims the benefit of German Patent application No. DE 10 2025 000 049.9 filed on Jan. 7, 2025, the disclosure of which is incorporated herein by reference in its entirety.

### FIELD OF THE TECHNOLOGY

Various embodiments relate to an injector for an implant, to an injector arrangement and to a method for producing an injector.

### SUMMARY

The focus here is on pharmaceutically active ophthalmological implants for treating diseases of the human eye. Such implants may be applied into a patient's eye by means of a special injector, which is often formed as a syringe with a tip in the form of a needle. In the eye, the implant is biologically degraded over time while releasing an active agent. For this purpose, the implant may for example have a polymer matrix which carries the active agent. The injector may be configured as a disposable injector and be supplied while already having the implant, so that the application of the implant into the eye can be carried out particularly efficiently and with little susceptibility to error by removing the injector from sterilised packaging and then inserting the injector into the eye. A part of the injector may nevertheless also be reusable and sterilisable. In order to eject the implant, the injector may have a feed mechanism, which in general can be actuated manually and with which the implant can be pushed through an injection opening.

One problem with such injectors is to ensure that the implant stays at its position in a storage portion during transport and when handling the injector, and does not slip into an unfavourable place in which the feed is impeded or prevented, or fall out of the injector or become damaged.

The above problem is solved by various features provided herein.

The focus can be on the idea that the injector may be provided with a holding element which retains the implant in the storage portion.

In detail, it is proposed that the implant is retained in the storage portion by means of a holding element which makes contact with the implant. The term “retained” means that the holding element holds the implant in position.

In various embodiments, the holding element is biocompatible. Due to the biocompatibility of the holding element, there is no cause for concern if the holding element does not remain entirely in the injector and is therefore implanted with the implant. Correspondingly, in various embodiments, the holding element may be at least partially destroyed when the implant is ejected and/or may remain adhering at least partially to the implant. The introduction of a part of the holding element into a patient is particularly unproblematic if the holding element consists of a material of which the implant also at least partially consists. Various embodiments include materials for the implant and the injector.

In various embodiments, the holding element may adhere to the implant and to the storage portion. Adhesion may be understood as a sufficiently strong static friction, which is adequate under usual accelerations of the injector during transport and the like because of the low mass of the implant. In various embodiments, however, the adhesion comprises a proportion of material bonding. It may therefore be said that the implant can be firmly bonded to the storage portion by the holding element. Accordingly, the holding element may be a drop of an adhesive material, or the like.

Various embodiments relate to the injector, and especially of a front portion which comprises the storage portion and the injection opening. The holding element may be introduced through an opening which is separate from the injection opening; for example, this makes it possible that the holding element is not arranged in the way of the implant and the implant therefore does not collide with the holding element, so that the ejection of the implant takes place reproducibly. In a particularly simple way, a rear opening of the front portion which is also employed for the feed mechanism may be used.

Also of interest in terms of production technology is the possibility of using a lateral opening of the front portion in order to introduce the holding element. The complexity of the manufacturing steps of the injector may thus be reduced, and in addition the holding element may be arranged partially outside a movement path of the implant and/or of the feed mechanism.

The front portion may be manufactured as a separate component, so that the implant can simply be introduced from behind before the injector is assembled.

The holding element may consequently be arranged on a rear portion of the implant.

Various embodiments relate to design features of the feed mechanism.

In various embodiments, an injector arrangement is provided.

It is proposed that the injector or the front portion is sterilely packaged.

All comments relating to the proposed injector may be referred to.

In various embodiments, a method for producing an injector is provided.

It is proposed that the implant and the holding element are introduced into the storage portion.

All comments relating to the proposed injector and the proposed injector arrangement may be referred to.

Various embodiments describe the way in which the holding element and the implant may be introduced into the storage portion. In order to introduce the holding element and/or the implant, according to various embodiments the aforementioned further opening may be used.

The holding element may have a liquid state during the introduction, and/or it may be actively cured after the introduction.

In various embodiments, the injector is an injector which is equipped and then sterilised by the manufacturer (disposable injector). Alternatively, a part of the injector may be equipped and sterilised by the manufacturer.

Various embodiments provide an injector for an implant, wherein the implant is stored in the injector, wherein the injector has an injection opening for delivering the implant into an organ, in particular an eye, a storage portion in which the implant is stored, and a feed mechanism for ejecting the implant from the storage portion through the injection opening, wherein the implant is retained in the storage portion by means of a holding element which makes contact with the implant.

In various embodiments, the holding element is biocompatible. In various embodiments, the holding element is at least partially destroyed when the implant is ejected, and/or in that the holding element remains adhering at least partially to the implant during the ejection, and/or in that the holding element consists, in particular entirely, of a material of which the implant also at least partially consists.

In various embodiments, the holding element consists at least partially of a polymer. In various embodiments, the polymer is polycaprolactone (PCL) or polylactic acid (PLA) or polyglycolic acid (PGA) or polylactic-co-glycolic acid (PLGA).

In various embodiments, the holding element adheres to the implant and to the storage portion. In various embodiments, the holding element adheres to the implant and/or to the storage portion by material bonding.

In various embodiments, the injector has a front portion, which is configured in particular as a hollow needle tip and which comprises the storage portion and the injection opening, in that the front portion has a further opening in addition to the injection opening, through which the holding element has been introduced. In various embodiments, the further opening is a rear opening of the front portion, through which the feed mechanism is moved, or in that the further opening is a lateral opening, which can subserve only the introduction of the holding element and which further can be closed by the holding element.

In various embodiments, the front portion is configured as a separate component. In various embodiments the injector has a main portion, in which the feed mechanism is displaceably guided and to which the front portion is fastened. In various embodiments, the main portion has a transport lock for the feed mechanism.

In various embodiments, the holding element adheres to the implant in a rear half, in particular a rear third, of the implant facing away from the injection opening. In various embodiments, the holding element adheres to a rear end of the implant.

In various embodiments, the feed mechanism has a feed element and a drive element, in that the drive element is manually adjustable from outside the injector and pushes the feed element forwards, in particular linearly, and in that the feed element engages with the implant from behind and pushes the implant forwards.

Various embodiments provide an injector arrangement having an injector as provided herein or having a front portion with an implant and a holding element for an injector as provided herein, wherein the injector or the front portion is sterilely packaged.

Various embodiments provide a method for producing an injector as described herein, wherein the implant and the holding element are introduced into the storage portion.

In various embodiments, the holding element is connected to the implant and the implant is subsequently introduced together with the holding element into the storage portion, or in that first the holding element and then the implant are introduced into the storage portion, or in that first the implant and then the holding element are introduced into the storage portion.

In various embodiments, the holding element and/or the implant is introduced into the storage portion through the further opening.

In various embodiments, the holding element is introduced into the injector and/or connected to the implant in a liquid state.

In various embodiments, the holding element is actively cured, in particular dried by UV and/or dried by means of a vacuum. In various embodiments, the holding element is actively cured through the further opening.

In various embodiments, the injector or the front portion is sterilised with the implant.

### BRIEF DESCRIPTION OF THE DRAWINGS

Various aspects are explained in more detail below with the aid of a drawing, which represents merely exemplary embodiments. In the drawing:

FIG. 1 shows a proposed injector with an ejected implant,

FIG. 2 shows sectional views of the injector in different states,

FIG. 3 shows possible ways of introducing the implant into the injector and retaining it there,

FIG. 4 shows further ways of introducing the implant into the injector and retaining it there, and

FIG. 5 shows a spring-based holding element.

### DETAILED DESCRIPTION

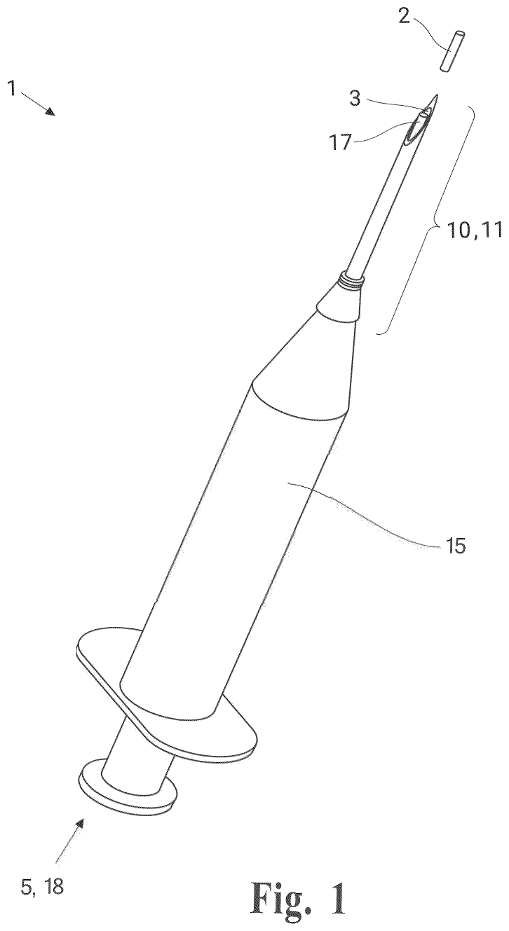

The exemplary embodiments which are represented in the figures, relate to an injector 1 for an implant 2. FIG. 1 shows an external view of such an injector 1, in which the implant 2 is represented already outside the injector 1. The implant 2, in various embodiments, is an ophthalmological implant 2 which can be pharmaceutically active. For example, the implant 2 may be a medicament which is injected into the eye, for example the vitreous humour. In the eye, the implant 2 is then degraded, in particular fully, while releasing an active agent. Accordingly, the implant 2 is, in some embodiments, fully biologically degradable. This degradation may last a few months, so that chronic, recurrent or in general persistent diseases may also be treated. Such implants 2 are used for example in the case of age-related macular degeneration, diabetic macular oedema or infections of the posterior segment of the eye.

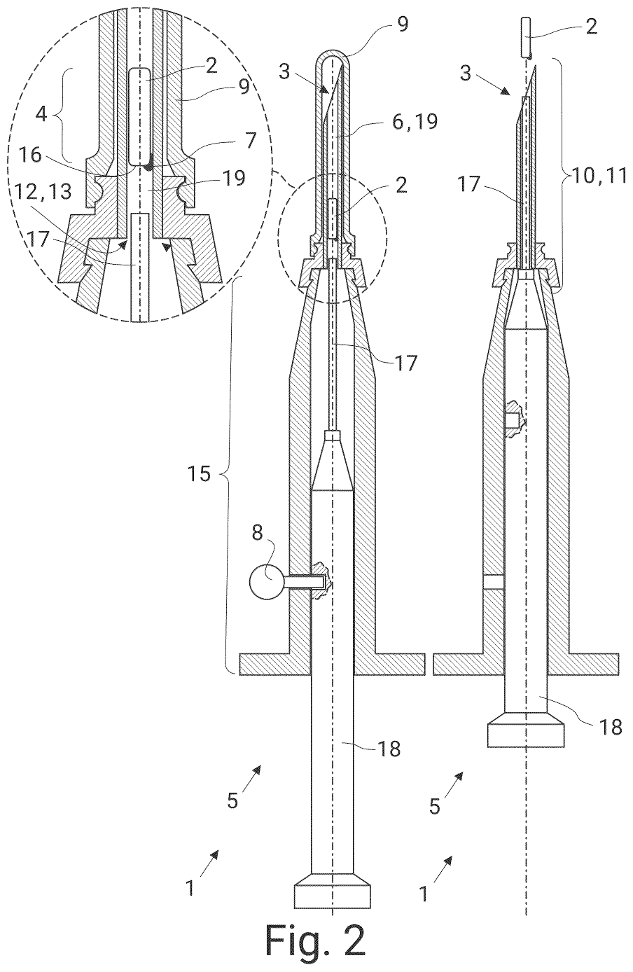

On viewing the injector 1, and for example FIG. 2, it can be seen that the implant 2 is stored in the injector 1. The injector 1 can be a disposable injector, so that the implant 2 has been introduced into the injector 1 by the manufacturer. The injector 1 has an injection opening 3 for delivering the implant 2 into an organ, in particular an eye, a storage portion 4 in which the implant 2 is stored, and a feed mechanism 5 for ejecting the implant 2 from the storage portion 4 through the injection opening 3. Overall, the injector 1 may be configured in the manner of a syringe so that the injection opening 3 can be inserted without prior incision into the organ, here the eye. After such insertion, the implant 2 is ejected by the feed mechanism 5 from the injection opening 3, which is then arranged in the organ. Depending on the embodiment, the feed mechanism 5 may already be actuated before the insertion into the organ in order to push the implant 2 forwards from the storage portion 4 and, in some embodiments, arrange it close to the injection opening 3.

The storage portion 4 can be at a distance from the injection opening 3 and may be connected to the injection opening 3 through a front channel portion 6. During use of the injector 1, the implant 2 is then conveyed, in particular pushed, by the feed mechanism 5 from the storage portion 4 through the front channel portion 6 and then ejected from the injection opening 3.

It is now proposed that the implant 2 is retained in the storage portion 4 by means of a holding element 7 which makes contact with the implant 2. In various embodiments, the holding element 7 makes contact with the implant 2 from the radial direction. The radial direction is oriented orthogonally to a longitudinal direction of the injector 1, along which the implant 2 is moved.

FIG. 2 shows on the left an injector 1 in the transport state and on the right an injector 1 with an ejected implant 2. In various embodiments, the injector 1 can have a transport lock 8, which in an active state prevents movement of the feed mechanism 5. Furthermore, in various embodiments, the injector 1 can have a protective cap 9 which covers the injection opening 3.

In various embodiments, the implant 2 is biocompatible. In various embodiments, the holding element 7 can be configured separately from the injector 1. The term “holding element” is to be interpreted broadly; in particular, the holding element 7 may be a drop of a material which connects the implant 2 materially to the storage portion 4 and therefore fulfils a similar function to an adhesive. It is however to be pointed out that the ejection of the implant 2 should not be substantially impeded. Nevertheless, the holding element 7 prevents the implant 2 from leaving the storage portion 4 at least for usual accelerations which occur during transport and/or handling of the injector 1, in combination with the low mass of the implant 2.

Due to the biocompatibility of the holding element 7, it is conceivable that the holding element 7 is at least partially destroyed when the implant 2 is ejected. Parts of the holding element 7 that are also injected into the organ are thus non-critical. This embodiment shows that simple retention of the implant 2 in the storage portion 4 is possible because of the biocompatibility, since it is not necessary to ensure that the holding element 7 remains in the injector 1.

It is therefore additionally or alternatively conceivable that the holding element 7 remains adhering at least partially to the implant 2 during the ejection.

Of particular interest is the possibility that the holding element 7 consists, in particular entirely, of a material of which the implant 2 also at least partially consists. There is thus also no concern in respect of the compatibility or the like of the holding element 7. It is nevertheless fundamentally possible that the holding element 7 is biologically inert and/or biologically degradable even if different materials are used than in the implant 2.

Further, in various embodiments, the holding element 7 consists at least partially of a polymer. In various embodiments, the polymer is polycaprolactone (PCL) or polylactic acid (PLA) or polyglycolic acid (PGA) or polylactic-co-glycolic acid (PLGA). One of these polymers may, as a constituent of the implant 2, form a matrix which carries a pharmaceutical product.

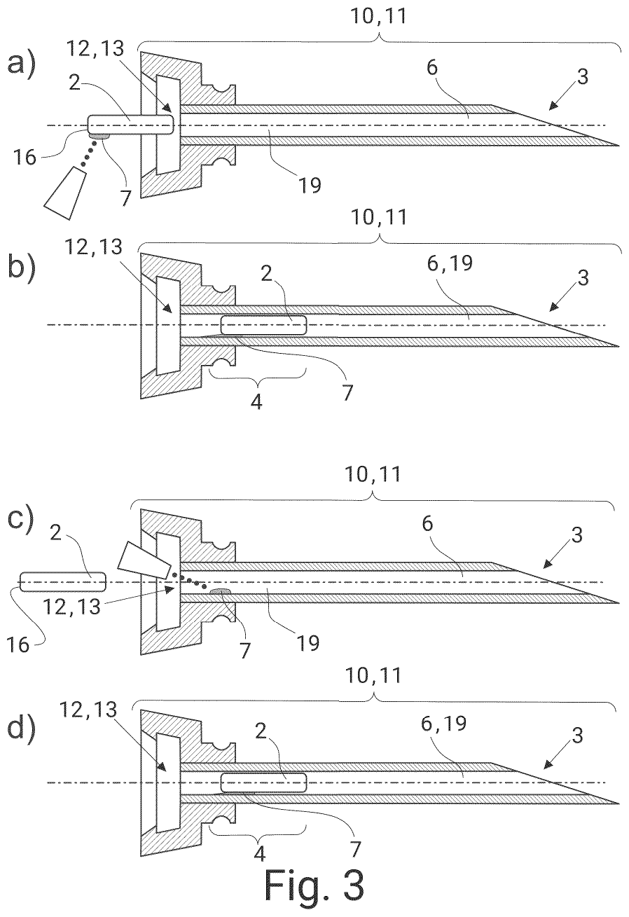

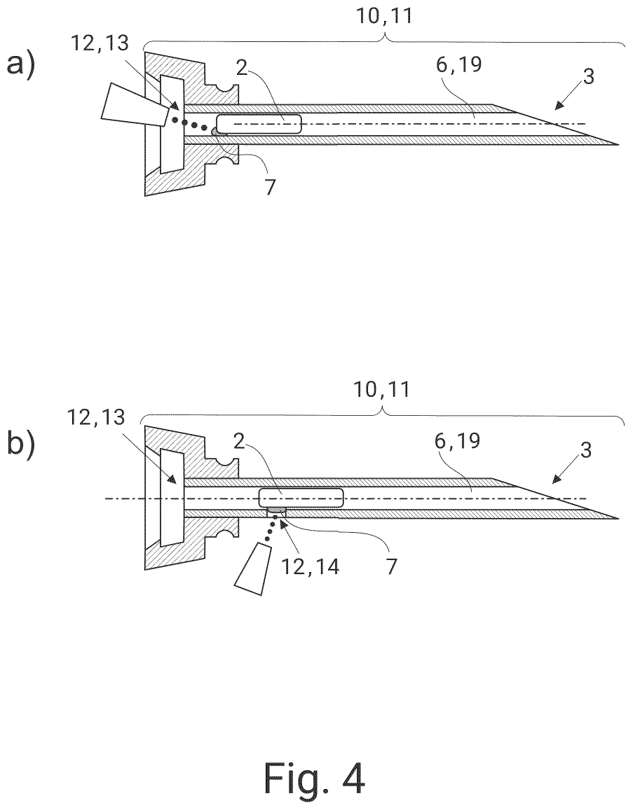

In various embodiments, the holding element 7 adheres to the implant 2 and to the storage portion 4. In various embodiments, the holding element 7 adheres to the implant 2 and/or to the storage portion 4 by material bonding. Overall, the holding element 7 may therefore as mentioned bond the implant 2 to the storage portion 4, the adhesion force needing to be balanced as mentioned between a holding effect and easy ejection of the implant 2. FIGS. 3 and 4 show several variants of the way in which the holding element 7 and the implant 2 may be introduced into the injector 1. These are explained in more detail below. Immediately of interest at this point is FIG. 4 b), which shows that the holding element 7 may readily also be retained on the storage portion 4 at least partially with a form fit. In this specific case, a combination of a form fit and material bonding is provided.

It is likewise conceivable that there is frictional interlocking between the holding element 7 and the implant 2 and/or between the holding element 7 and the storage portion 4, in particular without a form fit and/or without material bonding. In various embodiments, it is furthermore to be understood that the holding element 7 can be arranged in the storage portion 4 (FIGS. 2, 3, 4a) and 5).

In respect of the embodiment of the injector 1, the injector 1 may have a front portion 10 which is configured in particular as a hollow needle tip 11 and which comprises the storage portion 4 and the injection opening 3.

The front portion 10 may have a further opening 12 in addition to the injection opening 3, through which the holding element 7 has been introduced. In various embodiments, the further opening 12 is a rear opening 13 of the front portion 10, through which the feed mechanism 5 is moved (FIG. 3, FIG. 4a)).

Alternatively, the further opening 12 may be a lateral opening 14 which can subserves only the introduction of the holding element 7 and which further can be closed by the holding element 7 (FIG. 4b)). In general, it is of interest to provide a lateral opening 14 in the front portion 10 of the injector 1 since in this way the holding element 7 may be applied laterally with respect to the implant 2. The implant 2 is in general more readily accessible through the lateral opening 14, and a part of the holding element 7 may remain in the lateral opening 14. The feed mechanism 5 may then be pushed more favourably past the holding element 7. The lateral opening 14 may for example be a hole in the needle tip 11. The lateral opening 14 may be arranged in a rear half of the front portion 10. The lateral opening 14 may run obliquely, in particular orthogonally, to a longitudinal extent of the front portion 10.

A view of FIGS. 3 and 4 shows that, in various embodiments, the front portion 10 is configured as a separate component. It is thereby particularly readily possible to introduce the implant 2 into the front portion 10 through the rear opening 13 before assembling the injector 1. In various embodiments, the injector 1 further has a main portion 15 in which the feed mechanism 5 is displaceably guided and to which the front portion 10 is fastened. The fastening is represented as an enlargement in FIG. 2. Furthermore, as already mentioned, the main portion 15 may have a transport lock 8 for the feed mechanism 5.

The feed mechanism 5 and/or the front portion 10 may be made of steel or contain steel. The main portion 15 can be injection-moulded.

The figures reveal that the holding element 7 adheres to the implant 2 in a rear half, in particular a rear third, of the implant 2 facing away from the injection opening 3. In various embodiments, the holding element 7 adheres to a rear end 16 of the implant 2.

Further, the feed mechanism 5 may have a feed element 17 and a drive element 18, the drive element 18 may be manually adjustable from outside the injector 1 and push the feed element 17 forwards, in particular linearly, and the feed element 17 may engage with the implant 2 from behind and push the implant 2 forwards.

The injector 1 may have one channel 19 overall, which may be arranged in the front portion 10 and in the main portion 15. The channel 19 can contain a feed element 17 of the feed mechanism 5, which engages with the implant 2, and the implant 2. The channel 19 can open into the injection opening 3.

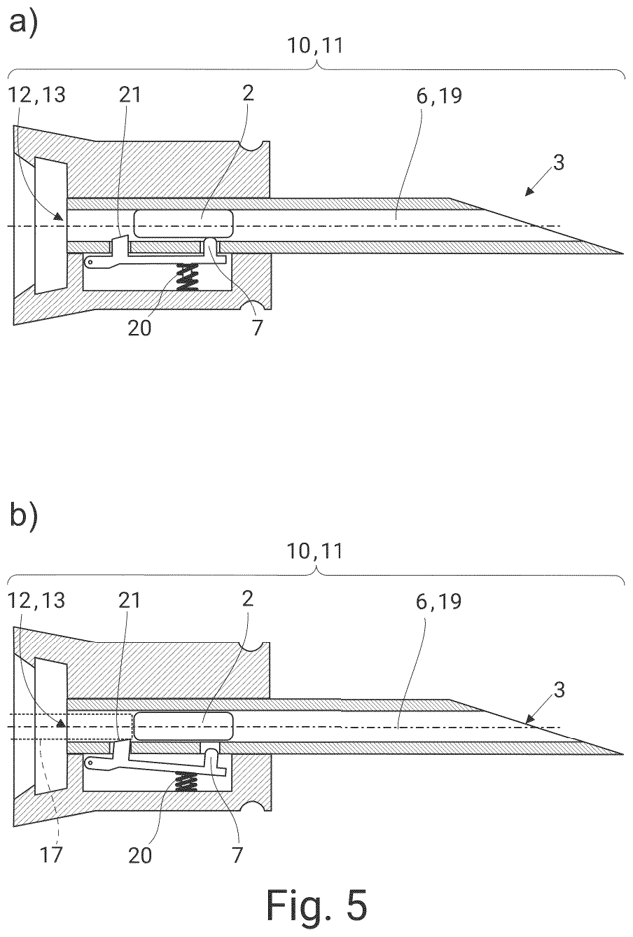

FIG. 5 shows an embodiment in which the holding element 7 is spring-loaded. In general, the holding element 7 may thus retain the implant 2 in the storage portion 4 by a spring force. The injector 1 may accordingly have a spring 20. In various embodiments, the holding element 7 is adjustable, in particular against the spring force, in order to eject and/or install the implant 2.

The holding element 7 may be adjusted by the feed mechanism 5, in particular before the feed mechanism 5 engages with the implant 2. This adjustment of the holding element 7 before the feed mechanism 5 engages with the implant 2 protects the implant 2 from damage, since the implant 2 can be adjusted while it is free from the holding element 7. Such a prior adjustment of the holding element 7 may also be provided independently of the spring loading. In principle, the holding element 7 may have a neutral position, in which the holding element 7 holds the implant 2, and be adjusted from the neutral position by the feed mechanism 5. As represented, the holding element 7 may have a chamfer 21 with which the feed mechanism 5 makes contact in order to adjust the holding element 7. FIG. 5a) shows an active setting of the holding element 7, and FIG. 5b) shows the way in which the holding element 7 is disengaged.

In order to install the implant 2, it is conceivable that the holding element 7 is manually adjusted or only brought into an active setting after the installation. In various embodiments, in this case the holding element 7 makes contact with the implant 2 in the front region. This prevents a broken implant 2 from falling forwards out of the injector. For the same reason, contact in the front region may also be provided for the holding element 7 of FIGS. 1 to 4. The holding element 7 may consist of plastic. Abrasion may thus be avoided, in contrast for example to a holding element 7 made of rubber. The holding element 7 may, as represented, be pivotable.

According to a further teaching, to which independent importance is attributed, an injector arrangement which has a proposed injector 1, or which has a front portion 10 with an implant 2 and a holding element 7 for a proposed injector 1, is proposed.

The injector 1 or the front portion 10 is sterilely packaged. The injector arrangement may accordingly have for example packaging which is sterilisable by steam and sterilised by steam or sterilised by ethylene oxide or sterilised with gamma radiation, in which the injector 1 or the front portion 10 is arranged. In the operating theatre, the injector 1 is then removed from the packaging and used, and, in some embodiments, subsequently disposed of. Alternatively, the front portion 10 is removed from the packaging and, in some embodiments, fitted onto a reusable main portion.

All comments relating to the proposed injector 1 may be referred to.

According to various embodiments, a method for producing a proposed injector 1 is provided.

During production, the implant 2 and the holding element 7 are introduced into the storage portion 4.

All comments relating to the proposed injector 1 and the proposed injector arrangement may be referred to.

FIGS. 3 and 4 show variants of the way in which the injector 1 may be produced. In one variant, which is represented in FIG. 3a) and b), the holding element 7 is connected to the implant 2 and the implant 2 is subsequently introduced together with the holding element 7 into the storage portion 4. For example, stronger adhesion may thus be achieved between the implant 2 and the holding element 7 than between the holding element 7 and the storage portion 4.

Alternatively, as represented in FIG. 3c) and d), first the holding element 7 and then the implant 2 may be introduced into the storage portion 4. This variant may lead to stronger adhesion between the storage portion 4 and the holding element 7. As a further alternative, and as represented in FIG. 4a), first the implant 2 and then the holding element 7 may be introduced into the storage portion 4. Similarly strong adhesion may thus be achieved. Ultimately, however, the adhesion also depends for example on the materials so that the order of the introduction is not the only influencing factor.

In various embodiments, the holding element 7 and/or the implant 2 are introduced into the storage portion 4 through the further opening 12.

It is fundamentally possible that the holding element 7 is introduced into the injector 1 and/or connected to the implant 2 in a liquid state.

Particularly in this case, but also in general, the holding element 7 may be actively cured, in particular dried by UV. The curing is also represented in the figures and may also be used for adapting the adhesion to requirements, regardless of whether or not the holding element 7 is also intended to be ejected. In various embodiments, the holding element 7 is actively cured through the further opening 12. The term “actively” refers to curing in which for example UV is used, and not to curing based purely on time. A vacuum may also be used in order to cure, in particular dry, the implant 2. Curing may also be achieved by heating the front portion 10.

Further, in various embodiments, the injector 1 or the front portion 10 is sterilised with the implant 2.

1. An injector for an implant, wherein the implant is stored in the injector, wherein the injector has an injection opening for delivering the implant into an organ, in particular an eye, a storage portion in which the implant is stored, and a feed mechanism for ejecting the implant from the storage portion through the injection opening,
wherein the implant is retained in the storage portion by means of a holding element which makes contact with the implant.

2. The injector according to claim 1, wherein the holding element is biocompatible.

3. The injector according to claim 1, wherein the holding element consists at least partially of a polymer.

4. The injector according to claim 1, wherein the holding element adheres to the implant and to the storage portion.

5. The injector according to claim 1, wherein the injector has a front portion, which is configured in particular as a hollow needle tip and which comprises the storage portion and the injection opening, in that the front portion has a further opening in addition to the injection opening, through which the holding element has been introduced.

6. The injector according to claim 5, wherein the front portion is configured as a separate component, preferably in that the injector has a main portion in which the feed mechanism is displaceably guided and to which the front portion is fastened.

7. The injector according to claim 1, wherein the holding element adheres to the implant in a rear half of the implant facing away from the injection opening, preferably in that the holding element adheres to a rear end of the implant.

8. The injector according to claim 1, wherein the feed mechanism has a feed element and a drive element, in that the drive element is manually adjustable from outside the injector and pushes the feed element forwards, and in that the feed element engages with the implant from behind and pushes the implant forwards.

9. An injector arrangement having an injector according to claim 1, wherein the injector is sterilely packaged.

10. A method for producing an injector according to claim 1,
wherein the implant and the holding element are introduced into the storage portion.

11. The method according to claim 10, wherein the holding element is connected to the implant and the implant is subsequently introduced together with the holding element into the storage portion, or
wherein first the holding element and then the implant are introduced into the storage portion, or
wherein first the implant and then the holding element are introduced into the storage portion.

12. The method according to claim 10, wherein the holding element and/or the implant is introduced into the storage portion through the further opening

13. The method according to claim 10, wherein the holding element is introduced into the injector and/or connected to the implant in a liquid state.

14. The method according to claim 10, wherein holding element is actively cured, in particular dried by UV and/or dried by means of a vacuum, further preferably in that the holding element is actively cured through the further opening.

15. The method according to claim 10, wherein the injector or the front portion is sterilised with the implant.

16. The injector according to claim 2, wherein the holding element is at least partially destroyed when the implant is ejected, and/or in that the holding element remains adhering at least partially to the implant during the ejection, and/or in that the holding element consists of a material of which the implant also at least partially consists.

17. The injector according to claim 3, wherein the polymer is polycaprolactone (PCL) or polylactic acid (PLA) or polyglycolic acid (PGA) or polylactic-co-glycolic acid (PLGA).

18. The injector according to claim 4, wherein the holding element adheres to the implant and/or to the storage portion by material bonding.

19. The injector according to claim 5, wherein the further opening is a rear opening of the front portion, through which the feed mechanism is moved, or in that the further opening is a lateral opening, which subserves only the introduction of the holding element and which further is closed by the holding element.

20. The injector according to claim 6, wherein the main portion has a transport lock for the feed mechanism.
